(19) United States
(12) Patent Application Publication
(10) Pub. No.: US 2005/0058700 A1
(43) Pub. Date: Mar. 17, 2005

(54) Preparations and methods for textile and/or fiber surface finishing, surface finished articles prepared thereby, and uses therefor

(76) Inventors: Rolf Wachter, Duesseldorf (DE); Manfred Weuthen, Langenfeld (DE); Claudia Panzer, Grevenbroich (DE); Erik Paff, Moenchengladbach (DE)

Correspondence Address: COGNIS CORPORATION;PATENT DEPARTMENT, 300 BROOKSIDE AVENUE, AMBLER, PA, 19002, US

(21) Appl. No.: 10/925,541

(22) Filed: Aug. 25, 2004

(30) Foreign Application Priority Data
Aug. 27, 2003 (DE) 103 39 358.7

(51) Int. Cl. A61K 9/127

(52) U.S. Cl. 424/450

(57) ABSTRACT

The invention relates to the use of liposomes, particularly liposomes based on soybean oil lecithins, for finishing textiles and fibers, a key aspect of the invention being that, besides a fiber-care effect, skin-friendly substances are transferred by liposome-finished fibers or textiles to the skin from those fibers or textiles.

### BACKGROUND OF THE INVENTION

The surface finishing of fibers or flat textiles with various chemicals is widely practised in the textiles field, for example for improving certain surface properties of textiles, such as their hydrophilia for example, or for providing the textiles and fibers with certain properties, for example with a crease-resistant finish or UV protection.

However, there is a need to finish textiles and fibers more intelligently, i.e. to provide them with properties which, hitherto, have not been accessible through known solutions.

The problem addressed by the present invention was to finish textiles and fibers in such a way that skin-friendly or skin-care substances could be transferred to the skin through the textiles or fibers.

### SUMMARY OF THE INVENTION

It has been found that the problem stated above can be solved by the use of liposomes. Accordingly, the present invention relates to the use of liposomes for the surface finishing of fibers and/or flat textiles.

Liposomes are generally spherical structures (typically 25 nm to 1 mm in diameter) of one or more concentric lipid double layers with a water-containing interior (lipid vesicles). These vesicular structures can be produced, for example, by mechanically distributing phospholipids (for example lecithin) very finely in aqueous media. Phospholipids are phosphoric acid diesters, occasionally monoesters, which may be regarded as lipids by virtue of their fat-like solubility properties attributable to the lipophilic and hydrophilic components and which, in the organism, are involved as membrane lipids in the formation of layer structures, the membranes. If they are suspended, they combine with one another to form ordered aggregates, such as micelles, lamellae, liposomes and other membrane structures. The fatty acid chains and aligned parallel to one another, as in liquid crystals, and the phosphoric acid ester groups point into the aqueous phase.

The liposomes preferably used in accordance with the invention, which are marketed under the name of Lipocutin®, are aqueous dispersions of stabilized liposomes optionally charged with active substance and based on soybean lecithin and cholesterol.

It is essential in the context of the present technical teaching that the liposomes contain a phospholipid layer based on hydrogenated triglycerides, preferably hydrogenated soybean lecithin.

The present invention also relates to a process for applying skin-friendly substances to the skin, the skin being contacted with liposome-containing fibers or textiles as described above and the liposomes of the fibers, or textiles being opened by mechanical pressure and/or heat, so that the dermatologically active substances and the phospholipids can be transferred to the skin.

### DEATILED DESCRIPTION OF THE INVENTION

Lecithins are glycerophospholipids formed by esterification from fatty acids, glycerol, phosphoric acid and choline. The naturally occurring lecithins, like the closely related kephalins, are derivatives of 1,2-diacylglycerol-3-phosphoric acids. The nature of the phosphoric acid is crucial to the later properties of the lecithins. The saturated fatty acid is normally esterified with the primary hydroxyl group of the glycerol and the unsaturated fatty acid with the secondary hydroxyl group thereof. Of crucial importance to the lecithins used in accordance with the invention is the fact that they are based on hydrogenated fatty acid triglycerides whereas the lecithins normally occurring in nature contain high levels of unsaturated fatty acids. Accordingly, the liposomes of the present invention are preferably free from unsaturated lecithins.

Soybean oil itself is a yellowish to brown-yellow, fatty, semidrying oil which is obtained from soybeans (Glycine max) or crushed soybeans by pressing and/or extraction with hydrocarbons (for example hexane). Oil content of the soybeans: 17-22%. The composition is shown in Table 1 below which sets out the fatty acid spectrum of soybean oil; the range of variation and the most common average value in % by weight of the total fatty acids are shown in parentheses. Table 1.

TABLE 1

| | | |
|---|---|---|
| C < 14 | <0.1 | |
| C14:0 | <0.5 | |
| C16:0 | 7.0-14 | (10) |
| C16:1 | <0.5 | |
| C18:0 | 1.4-4.5 | (4.5) |
| C18:1 | 19-30 | (21) |
| C18:2 | 44-62 | (56) |
| C18:3 | 4-11 | (8) |
| C20:0 | <1.0 | (0.5) |
| C20:1 | <1.0 | (0.5) |
| C22:0 | <0.5 | |

55 to 65% of the total fatty acids of soybean oil are polyunsaturated fatty acids. The sterol content of soybean oil is on average 0.37% (of which cholesterol 0.3-0.5%). Besides cholesterol, soybean oil contains above all ergost-5-en-3b-ol, campesterol and sitosterol. The sterol content can be reduced by ca. 30% by refining. Soybean oil also contains free fatty acids, lecithin and up to 0.8% tocopherol. Soybean oil has a density of 0.916 to 0.922, a melting point of −15 to −8° C., a solidus point of 282° C., a saponification value of 188 to 195, an iodine value of 120 to 136 and an acid value of 0.3 to 3.0; the content of unsaponifiables is typically 0.5 to 1.5%.

In addition, the liposomes according to the invention preferably contain cholesterols. Cholesterin, systematically 5-cholesten-3b-ol, or cholesterol: empirical formula: C₂₇H₄₆O, molecular weight 386.66; forms completely colorless flakes, density 1.052, melting point 148.5° C., boiling point 360° C. (slight decomposition). It is a compound which is virtually insoluble in water, soluble in alcohol only sparingly when cold, but more readily after heating and soluble in ether, benzene and petroleum ether. In the liposomes of the present invention, it occurs in quantities of preferably around 50% by weight. Preferably, the liposomes of the present invention also contain alkyl phosphates, preferably dialkyl phosphates and, in particular, dicetyl phosphate.

The liposomes of the present invention preferably have a diameter of 25 nm to 1 mm; their mean particle size is in the range from 100 to 800 nm, preferably in the range from 100 nm to 500 nm and more particularly in the range from 150 to 300 nm. A key advantageous aspect of the present technical teaching is that the liposomes as described above preferably contain skin-care ingredients in their interior. The liposomes of the present invention typically enclose a water-containing core which is capable of accommodating a number of ingredients known to the expert. These ingredients may be skin-care substances of all kinds providing they are capable of being stably enclosed by liposomes in a water-containing environment. Examples of such ingredients are protein hydrolyzates, moisturizers, polymers and, in particular, perfume oils. Hydrophilic, water-dispersible substances in particular are suitable for “encapsulation” in the liposomes. Lipophilic substances may also be integrated in the liposomes, although they are contained in the membrane and not in the water-filled interior. However, the liposomes of the present invention must be free from anionic compounds.

The liposomes of the present invention may advantageously be used in the form of water-containing preparations containing the liposomes and preferably at least one solubilizer preferably selected from the groups of C₂₋₆ monoalcohols. A particularly suitable alcohol for the purposes of the present invention is ethanol. Corresponding preparations typically contain 95 to 50% by weight water and 1 to 50% by weight of the alcohol. The liposomes are present in quantities of 0.1 to 15% by weight, preferably 1 to 5% by weight and more particularly 1 to 2.5% by weight. A typical preparation according to the present invention contains 60 to 80% by weight water, 10 to 25% by weight of a lower organic alcohol, preferably ethanol, and 5 to 15% by weight liposomes. However, purely aqueous preparations are also possible. The liposomes are generally finely dispersed in the continuous aqueous phase.

Demineralized water is preferably used for producing the preparations. Typical preservatives, preferably phenoxyethanol, may be used to protect the preparations. Besides the liposomes, the preparations may also contain other ingredients, preferably surfactants and fabric softeners, for example quaternary ammonium compounds. If the liposomes of the present invention are to be formulated together with surfactants, it can be of advantage not to use anionic surfactants. However, cationic and/or nonionic surfactants are preferably used.

The water-containing preparations according to the invention have pH values of 4.5 to 7.0, preferably in the range from 5 to 7 and more particularly in the range from 5.5 to 6.5.

The present invention also relates to a process for applying skin-friendly substances to the skin, the skin being contacted with liposome-containing fibers or textiles as described above and the liposomes of the fibers or textiles being opened by mechanical pressure and/or heat, so that the dermatologically active substances and the phospholipids can be transferred to the skin. The phospholipids from the liposomes can increase the penetration of skin-care substances and thus have an additional use. In practice, this means that, on heating to the temperature of the skin or higher, i.e. >35° C., textiles or fibers in the context of the present invention undergo a drying process in which the liposomal structure is broken up and the ingredients are able to pass in fine distribution to the surface of the fibers. Without wishing to be confined to any particular theory, applicants assume that the liposomes thus open and a thin film of the ingredients of the liposomes and the phospholipids is formed on the fibers. This film on the fibers comes into contact with the skin and is then transferred by mechanical processes.

The finishing of fibers and/or flat textiles in accordance with the invention opens up a number of potential applications. The finishing of the fibers or textiles itself may be carried out in known manner, i.e. for example by a spray or immersion treatment. However, the liposomes may also be used in automatic washing processes, for example by “flushing” into a domestic washing machine.

Textiles surface-finished with liposomes in accordance with the present invention are capable of providing the wearer with a range of positive properties, more particularly skin-care effects, a key positive aspect being that, by virtue of their very fine distribution, the liposomes and their ingredients do not leave the user with a greasy impression. By virtue of their low concentration, the active ingredients penetrate rapidly into the skin where they are able to develop their skin-care effects quickly and effectively. The continuous transport of skin-care substances from the liposomes onto the skin provides for a long-lasting effect by comparison with direct application of the skin care substances. Another aspect is that, by virtue of the moisture naturally present on the surface of the skin in conjunction with suitably adapted ingredients of the liposomes, for example by the addition of emulsifiers to the skin-care ingredients, emulsions are formed on the surface of the skin of the person wearing the textiles, which provides for a range of formulations for skin-care ingredients.

The liposomes described above are particularly suitable for the finishing of fibers and/or textiles completely or partly containing wool, cotton, silk, cellulose and/or synthetic fibers, and also for the finishing of paper. They are particularly suitable for the finishing of cotton, cotton/wool blends and also synthetic fibers, preferably nylon fibers.

### EXAMPLES

An aqueous liposome dispersion (Plantatex® Lip, Cognis) containing 2% by weight lipids was used. The liposomes consisted of hydrogenated soybean lecithin, cholesterol and dicetyl phosphate. The mean particle size was 400 nm. The aqueous liposome dispersion had a pH of 6.5.

Application Tests

1. Pump Spray

An aqueous preparation containing 10% by weight of the aqueous liposome dispersion and 0.4% by weight phenoxyethanol (balance to 100% by weight water) was prepared. This preparation had a viscosity of <50 mPas and a pH of 6.6 The preparation was stable in storage for at least 12 weeks at −5, 8, 23 and 40° C. The preparation was applied as a pump spray 20 times to a 20×30 cm large test fabric (cotton Wfk10A or wool Wfk60A or knitted cotton Wfk80A). Tests were also carried out on silk 70A and Lenzing viscose. The test fabric was dried at room temperature. Table 2 shows the content of liposomes on the test fabrics.

TABLE 2

| | Weight | | | Liposome |
|---|---|---|---|---|
| | before | Weight after | Quantity of | content |
| | application | application | formulation | mg/cm² |
| Cotton Wfk10A | 7.38 | 9.50 | 2.12 | 0.007 |
| Wool Wfk60A | 9.31 | 12.29 | 2.98 | 0.010 |
| Knit Wfk80A | 10.64 | 13.72 | 3.08 | 0.010 |

The liposomes were then eluted from the test fabrics with water, the liposomes being detectable in the aqueous eluate.

These preparations were sprayed onto textiles of cotton Wfk10A. The sensory properties of the textiles (for example sheen, softness, elasticity, wearing comfort, drying behavior, hardness) of the textiles were tested against the untreated textile (i.e. textile treated with water only) by a group of 10 examiners. The textiles treated in accordance with the invention had better elasticity, hardness and softness properties than the textile treated solely with water.

2. Care Balm

Care balm is the term used for formulations which can be flushed in from the dispensing drawer of a washing machine in order to apply the ingredients to the textiles during the washing process. Two formulations were tested. Besides the aqueous liposome dispersion, they contained commercially available softeners based on commercially available quaternary ammonium compounds (Dehyquart® AU, Cognis). The formulation is shown in Table 3:

TABLE 3

| | Formulation A, % by weight |
|---|---|
| N-Methyl-N,N-bis-(acyloxyethyl)-N-(2- | 2 |
| hydroxyethyl)-ammonium methosulfate | |
| Phenoxyethanol | 0.4 |
| Liposome dispersion | 10 |
| Water | Balance |

The formulations were prepared by stirring the quaternary ammonium compounds (QUATS) into warm water and then adding the liposome dispersion at 30° C. The formulations had a pH of 4.3 and a viscosity of <50 mPas.

The preparations of Table 2 were applied in the softener cycle of the wash program of a commercially available domestic washing machine (Miele W 467). Quantities of 80 g of the above formulations pet dispensing cycle were applied to the test fabric of cotton Wfk10A or cotton knit 80A. The liposomes could then be detected on the textiles.

For the sensory evaluation, a terry test fabric was finished with the liposomes as described above. This fabric was compared by a panel of 11 examiners with a terry cloth which had been finished with formulation A, but without the liposome component (reference). The QUAT content was 4% by weight. In direct comparison, the textile finished in accordance with the invention was awarded far better scores than the comparison cloth in regard to drying behavior.

In another test, a 10% by weight aqueous liposome dispersion was applied by immersion to nylon stockings which were then dried in air. 0.14% by weight liposomes (based on the active substance used) were detected on the stockings by reweighing. In an evaluation by a panel of 11 examiners, the stockings finished in accordance with the invention were found to be softer and more comfortable to wear than the comparison stockings treated solely with water.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method comprising: (a) providing a substrate selected from the group consisting of fibers and textiles; and (b) contacting the substrate with a liposome, wherein the liposome comprises a hydrogenated triglyceride-containing phospholipid layer.

2. The method according to claim 1, wherein the hydrogenated triglyceride-containing phospholipid layer comprises a hydrogenated soybean lecithin.

3. The method according to claim 1, wherein the liposome further comprises cholesterol.

4. The method according to claim 1, wherein the liposome further comprises a phosphate selected from the group consisting of alkyl phosphates and dialkyl phosphates.

5. The method method according to claim 4, wherein the phosphate comprises dicetyl phosphate.

6. The method according to claim 1, wherein the liposome further comprises a dialkyl phosphate.

7. The method according to claim 1, wherein the liposome has a diameter of from 25 nm to 1 mm.

8. The method according to claim 1, wherein the liposome further comprises a skin-care ingredient.

9. The method according to claim 8, wherein the skin-care ingredient is water-dispersible and is encapsulated within the liposome.

10. The method according to claim 8, wherein the skin-care ingredient is lipophilic.

11. A preparation comprising water, a solubilizer and a liposome, wherein the liposome comprises a hydrogenated triglyceride-containing phospholipid layer.

12. The preparation according to claim 11, wherein the hydrogenated triglyceride-containing phospholipid layer comprises a hydrogenated soybean lecithin.

13. The preparation according to claim 1 1, wherein the solubilizer comprises an organic C₂₋₆ monoalcohol.

14. The preparation according to claim 11, wherein the liposome further comprises cholesterol.

15. The preparation according to claim 11, wherein the liposome further comprises a phosphate selected from the group consisting of alkyl phosphates and dialkyl phosphates.

16. The preparation according to claim 11, wherein the liposome has a diameter of from 25 nm to 1 mm.

17. A process comprising: (a) providing a substrate treated according to the method of claim 1; and (b) contacting at least a portion of an animal's skin with the substrate, whereby the liposome passes from the substrate to the portion of the skin.

18. The process according to claim 17, wherein the hydrogenated triglyceride-containing phospholipid layer comprises a hydrogenated soybean lecithin.

19. The process according to claim 17, wherein the liposome further comprises a phosphate selected from the group consisting of alkyl phosphates and dialkyl phosphates.

20. The process according to claim 17, wherein the liposome further comprises a skin-care ingredient.
